(19) United States
(12) Patent Application Publication
(10) Pub. No.: US 2011/0238158 A1
(43) Pub. Date: Sep. 29, 2011

(54) EXPANDABLE VASCULAR ENDOLUMINAL PROSTHESES

(75) Inventors: James A. Heringes, Monmouth Junction, NJ (US); John Kula, Birdsboro, PA (US); Ronald Rakos, Neshanic Station, NJ (US)

(73) Assignee: Prescient Medical, Inc., Doylestown, PA (US)

(21) Appl. No.: 13/155,300

(22) Filed: Jun. 7, 2011

Related U.S. Application Data
(63) Continuation of application No. 11/822,336, filed on Jul. 5, 2007, now abandoned.
(60) Provisional application No. 60/851,755, filed on Oct. 16, 2006.
(60) Provisional application No. 60/818,508, filed on Jul. 6, 2006.

(51) Int. Cl. A61F 2/82

(52) U.S. Cl. 623/1.16

(57) ABSTRACT

The invention provides expandable tubular endoluminal prostheses for the treatment of atherosclerotic lesions of blood vessels, including vulnerable plaque lesions, and methods of treatment using the prostheses. Various prostheses of the invention are characterized by hoop strength suitable for treating vulnerable plaque lesions, good conformability and good apposition to vessel walls, as well as minimal coverage areas in order to minimize the inflammatory response to the implanted prostheses.

This application is a Continuation of U.S. application Ser. No. 11/822,336, filed Jul. 5, 2007, which claims the benefit of U.S. Provisional Application Nos. 60/851,755, filed Oct. 16, 2006 and 60/818,508, filed Jul. 6, 2006, each of which is incorporated herein by reference in their entirety.

### FIELD OF THE INVENTION

The invention relates generally to the fields of expandable vascular endoluminal prostheses and their use in treating atherosclerotic lesions.

### BACKGROUND OF INVENTION

Vulnerable plaques, which are sometimes known as high-risk atherosclerotic plaques, are arterial atherosclerotic lesions characterized by a subliminal thrombotic lipid-rich pool of materials contained by a thin fibrous cap. Although vulnerable plaques are non-stenotic or nominally stenotic, it is believed that their rupture, resulting in the release of thrombotic contents, accounts for a significant fraction of adverse cardiac events.

U.S. Publication No. 2002/0004679 discloses drug-eluting polymer stents for treating restenosis with topoisomerase inhibitors, and is incorporated herein by reference in its entirety.

U.S. Publication No. 2003/0125799 discloses intravascular stents for the treatment of vulnerable plaque that consist of opposing end ring portions and a central strut portion having a zig-zag configuration that connects with the end portion at apices of the zig-zag structure, and is incorporated herein by reference in its entirety.

U.S. Publication No. 2005/0137678 discloses a low-profile resorbable polymer stent and compositions therefore, and is incorporated herein by reference in its entirety.

U.S. Publication No. 2005/0287184 discloses drug-delivery stent formulations for treating restenosis and vulnerable plaque, and is hereby incorporated by reference herein in its entirety.

### SUMMARY OF INVENTION

The present invention provides tubular endoluminal prostheses, and related methods, for treating atherosclerotic lesions, such as vulnerable plaques.

One embodiment of the invention provides an expandable, at least substantially tubular, intravascular prosthesis that includes circumferential sinusoidal members connected by at least substantially linear longitudinal struts.

One embodiment of the invention provides an expandable, at least substantially tubular, intravascular prosthesis that includes circumferential undulating sinusoidal members connected by at least substantially linear longitudinal struts.

One embodiment of the invention provides an expandable, at least substantially tubular, intravascular prosthesis that includes longitudinally oriented sinusoidal members connected by at least substantially sinusoidal, partly longitudinally-traversing strut members.

One embodiment of the invention provides an expandable, at least substantially tubular, intravascular prosthesis that includes longitudinally oriented at least substantially sinusoidal members connected by at least substantially straight, partly longitudinally-traversing struts members.

One embodiment of the invention provides an expandable, at least substantially tubular, intravascular prosthesis that includes longitudinally oriented, at least substantially sinusoidal members connected by at least substantially sinusoidal radial struts.

One embodiment of the invention provides an expandable, at least substantially tubular, intravascular prosthesis that includes compressed elliptical shaped (“hourglass-shaped”) cells disposed at an angle to the longitudinal axis of the prosthesis interconnected by longitudinal and circumferentially oriented struts. The cells may also be interconnected by a single bend or “s” shaped struts that are diagonally oriented with respect to the longitudinal axis of the prosthesis.

One embodiment of the invention provides an expandable, at least substantially tubular, intravascular prosthesis that includes at least substantially X-shaped elements interconnected by smaller at least substantially sinusoidal connecting elements

A further embodiment of the invention provides a method for treating an atherosclerotic vascular lesion, such as a vulnerable plaque, in a patient in need thereof, comprising the step of: deploying a prosthesis according to the invention at the site of the lesion in a blood vessel of the patient. The site may, for example, be in a coronary artery. The prosthesis may be covered or uncovered. The prosthesis may be coated or uncoated.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A shows an embodiment of a prosthesis according to the invention that includes circumferential sinusoidal members connected by linear longitudinal struts.

FIG. 1B shows a close-up view of the structure of the embodiment of FIG. 1A.

FIG. 2A shows an embodiment of a prosthesis according to the invention that includes circumferential undulating sinusoidal members (for good surface area coverage) connected by linear longitudinal struts for column strength required for loading into the delivery system and for accurate deployment without jumping.

FIG. 2B shows a close-up view of the structure of the embodiment of FIG. 2A.

FIG. 3A shows an embodiment of a prosthesis according to the invention that includes longitudinally oriented sinusoidal members (to promote endothelialization) connected by sinusoidal longitudinally-traversing struts members for stability and support.

FIG. 3B shows a close-up view of the structure of the embodiment of FIG. 3A.

FIG. 3C shows a close-up view of an embodiment of a prosthesis according to the invention that includes longitudinally oriented sinusoidal members (to promote endothelialization) connected by straight longitudinally-traversing struts members for stability and support and to add column strength. This provides a tighter cell structure for enhanced flexibility.

FIG. 3D shows a close-up view an embodiment of a prosthesis according to the invention that includes longitudinally oriented sinusoidal members (to promote endothelialization) connected by sinusoidal radial struts. This geometry creates a closed cellular structure for enhanced vessel apposition and evenly distributed radial force.

FIG. 4A shows an embodiment of a prosthesis according to the invention that includes compressed elliptical shaped (“hourglass-shaped”) cells disposed at an angle to the longitudinal axis of the prosthesis interconnected by longitudinal and circumferentially oriented struts (to provide flexibility and radial strength). The hourglass-shaped elements and connecting bars provide excellent column strength.

FIG. 4A shows a close-up view of the structure of the embodiment of FIG. 4B.

FIG. 5A shows an embodiment of a prosthesis according to the invention that includes X-shaped elements interconnected by smaller sinusoidal connecting elements. The sinusoidal elements minimize foreshortening during radial expansion of the prosthesis from a compressed delivery configuration to its deployed state.

FIG. 5B shows a close up view of a section of the prosthesis structure of the embodiment of FIG. 5A.

FIG. 6 shows a section of an embodiment of a prosthesis according to the invention that includes sinusoidal ring sections for radial support (see Detail D) interconnected by lateral sinusoidal struts having 6 bends (see Detail A) positioned on an angle from the longitudinal axis. The lateral sinusoidal struts provide column strength and minimize foreshortening.

FIG. 7 shows a portion of an embodiment of a prosthesis that is similar to the embodiment shown in FIG. 6. The lateral sinusoidal struts are wider in this embodiment to provide additional column strength for loading into the delivery system and for accurate deployment without jumping.

FIG. 8 shows a section an embodiment of a prosthesis according to the invention that includes circumferential undulating sinusoidal members connected by linear longitudinal struts. The embodiment of FIG. 8 is related to the embodiments of FIGS. 2A and 2B. The increased number of linear longitudinal struts provides higher column strength for loading into the delivery system and for accurate deployment without jumping.

FIG. 9 shows a section of an embodiment of a prosthesis according to the invention that has nested cruciform shaped cells that are formed from lateral (along the longitudinal axis of the prosthesis) sinusoidal elements interconnected by staggered transverse sinusoidal connecting struts having two bends. The structure provides high column strength while simultaneously allowing for adequate radial strength for minimal vessel trauma and good vessel apposition. Radial force can be balanced accurately within this design by adjusting the sinusoidal strut patterning and thickness. Coverage area can also be adjusted to provide less metal surface and a more open structure for side branch access.

FIG. 10 shows an embodiment of a prosthesis according to the invention that includes compressed elliptical shaped (“hourglass-shaped”) cells disposed at an angle to the longitudinal axis of the prosthesis in which the ends of adjacent hour-glass shaped elements are connected by straight struts and each hourglass-shaped element is connected at its side to one transversely adjacent hourglass-shaped element by a sinusoidal connecting element. The “s-shaped” struts allow the hourglass shapes to fold into each other for easier delivery system loading. When unfolding during device deployment they ensure that the prosthesis does not jump forward in the vessel. The hourglass-glass shapes provide a web like structure to maximize cell growth over the thin cap of the vulnerable plaque.

FIG. 11 (flat pattern) shows a portion of an embodiment of a prosthesis that is similar to the embodiment shown in FIG. 6.

FIG. 12 (isometric view) shows a portion of an embodiment of a prosthesis that is similar to the embodiment shown in FIG. 6.

FIGS. 13 (flat pattern) and 14 (isometric view) show a portion of an embodiment of a prosthesis that is similar to the embodiment shown in FIG. 6. This structure again has a more open design, providing a coverage (prosthesis wall member area/total tubular area) of approximately 11% (in its expanded state). A hinge feature has been added to this embodiment (see Detail A). The hinge feature allows the structure to collapse to an even smaller diameter for loading into the delivery system.

### DETAILED DESCRIPTION

The invention provides tubular endovascular prostheses for the treatment of atherosclerotic lesions and vulnerable plaques in particular, as well as methods of treatment using the prostheses.

The prostheses of the invention are preferably expandable so that their radii can be increased to contact the wall of blood vessel. The prosthesis may be balloon-expandable and/or self-expanding. In one embodiment, the prosthesis is balloon expandable at a pressure of 3 ATMs or less. In another embodiment, the prosthesis is self-expanding by virtue of being composed of a shape-memory metal alloy or a shape-memory polymer.

For vulnerable plaque applications, the endoluminal prostheses of the present invention do not need the hoop strength and radial resiliency that is required by conventional stents that are used in conjunction with angioplasty procedures to prevent restenosis. Accordingly, the prostheses of the invention may have or lack such hoop strength, and may be of a lighter construction than conventional stents. In addition, various prostheses of the invention are characterized by excellent conformability and excellent apposition to vessel walls, two traits that are desirable for treating vulnerable plaque lesions. This is accomplished with minimal radial force being applied to the vessel wall to minimize vessel trauma. The wall thickness of prosthesis according to the invention may be made quite thin in order to maximum the lumen area when deployed and thereby prevent or minimize any potential thrombosis. In one embodiment, the wall thickness of the shield is 0.0025 inches or less to minimize thrombosis. While not being limited by theory, Applicants believe that the prostheses of the invention can passivate vulnerable plaque lesions as a result of stimulating the growth and/or migration of endothelial cells to cover the lumen-side wall area of the prostheses, thereby also covering the subject lesion (“endothelialization”).

Various aspects of the invention are described below with reference to the appended figures.

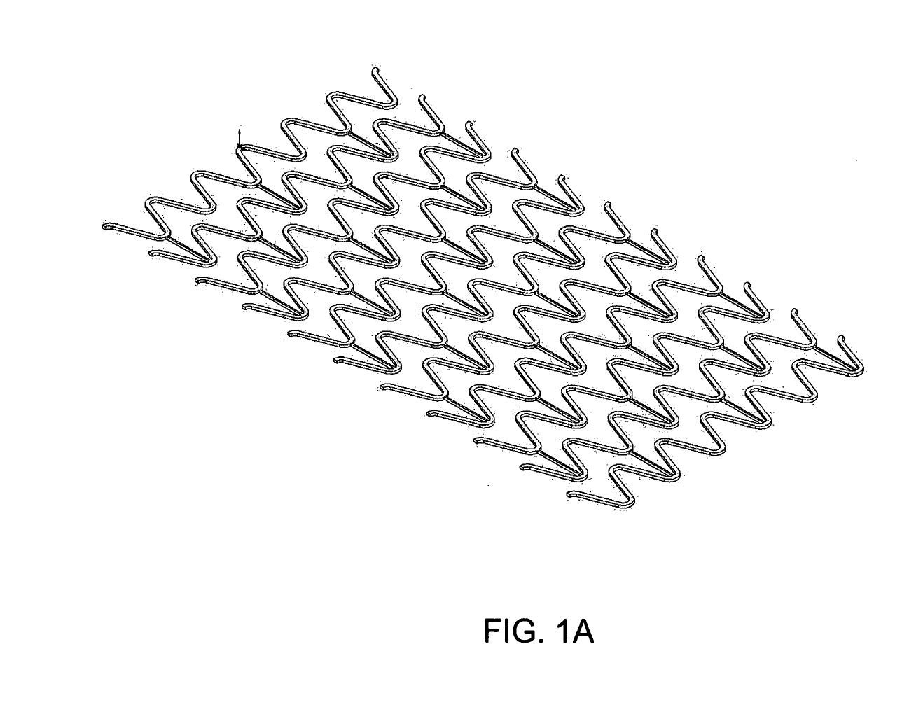

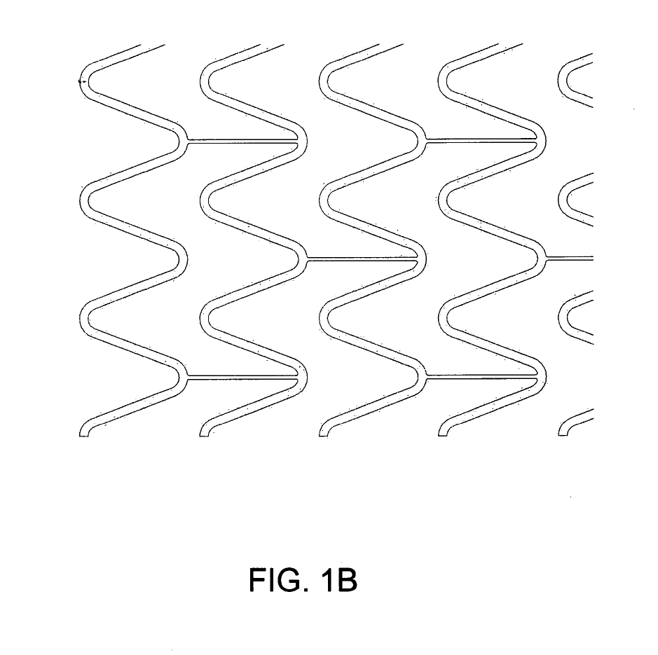

FIG. 1A shows an embodiment of a prosthesis according to the invention that includes circumferential sinusoidal members connected by linear longitudinal struts. The view shown in FIG. 1A is a schematic “rolled-out,” flattened view of the tubular configuration. FIG. 1B shows a close-up view of the structure of the embodiment of FIG. 1A. The design may have relatively low radial force, for example, exerting about 240 mm of Hg, in order to minimize trauma and/or distension of a treated blood vessel such as an artery. In one version of the embodiment, the prosthesis has a wall thickness in the range of 0.0025-0.0035 inches, or approximately 64-90 microns, a typical strut width of about 0.005 inches or about 130 microns, typical openings (in the wall of the prosthesis) of around 500 microns, a largest potential opening (for side branch access) of about 2.1 mm and coverage (prosthesis wall member area/total tubular area) of 17% (in its expanded state).

Accordingly, one embodiment of the invention provides a stent or tubular endoluminal prosthesis for the treatment of an atherosclerotic lesions, such as a vulnerable plaque, that includes: a plurality of radial sinusoidal bands each sinusoidal band comprising peaks and troughs, wherein the peaks and troughs of laterally neighboring bands are in-phase; and a plurality of lateral connector elements connecting neighboring bands to each other, wherein the lateral connector elements connect alternate peaks of a band to the neighboring trough of a neighboring band and wherein the lateral connector elements are alternately placed laterally. The lateral connector elements may, for example, be or include at least substantially straight bars.

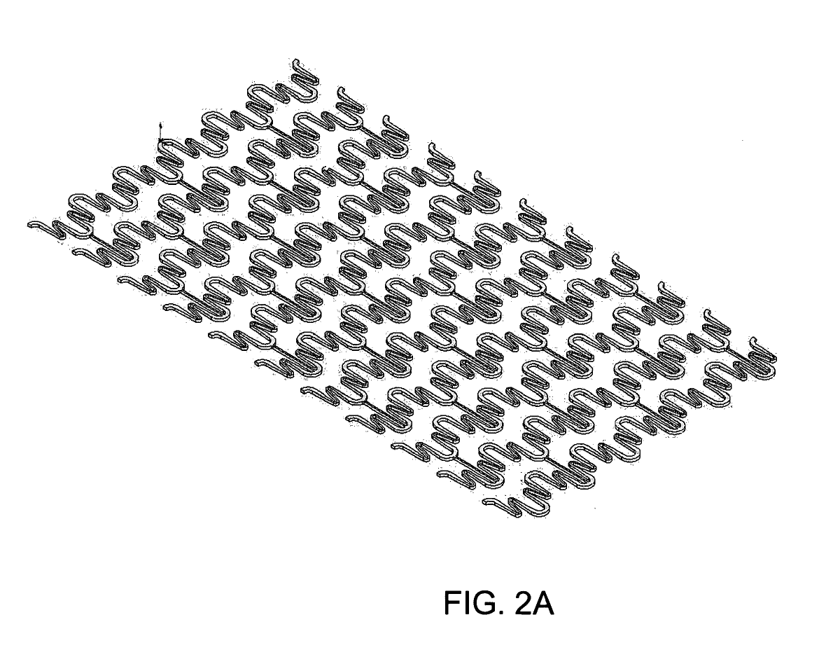

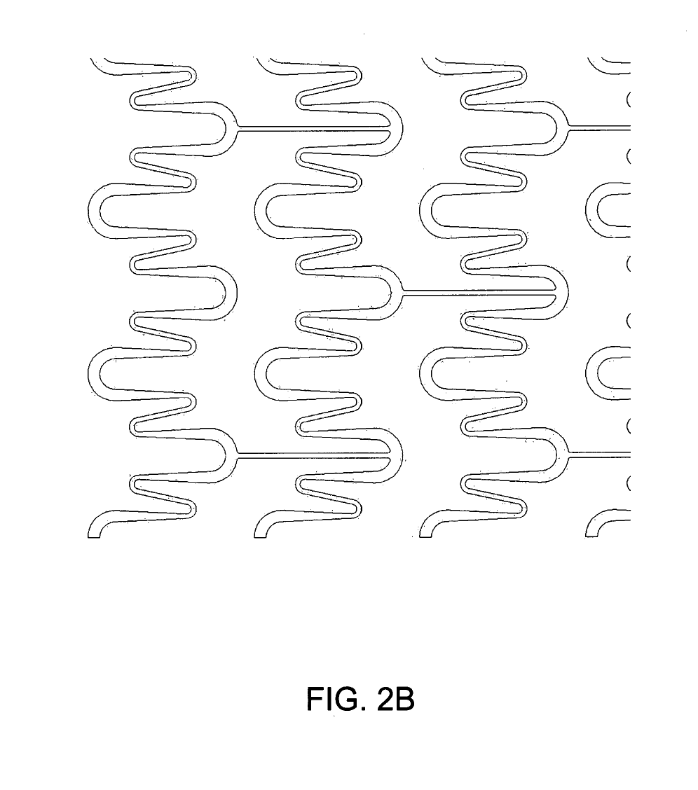

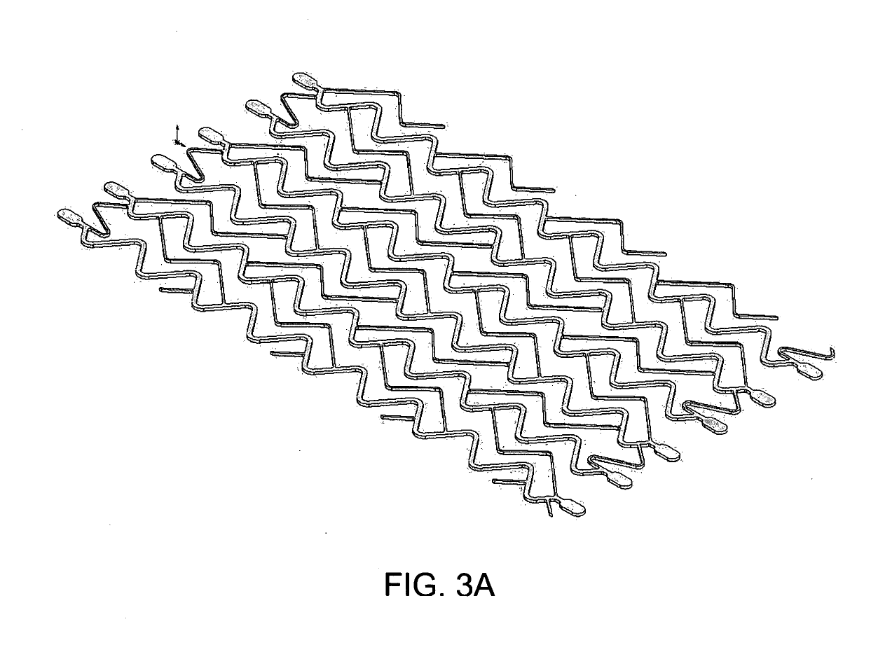

FIG. 2A shows an embodiment of a prosthesis according to the invention that includes circumferential undulating sinusoidal members (for adequate surface area coverage) connected by linear longitudinal struts. The view shown in FIG. 2A is a schematic “rolled-out,” flattened view of the tubular configuration. FIG. 2B shows a close-up view of the structure of the embodiment of FIG. 2A. This prosthesis design is very flexible with its longitudinal undulations. The design is characterized by good conformability and wall apposition in a blood vessel. The design may have low radial force, for example, exerting about 50-200 mm of Hg, such as 60-70 mm of Hg, in order to minimize trauma and/or distension of a treated blood vessel such as an artery. In comparison, marketed self-expanding stents designed for stenotic disease typically exert forces in the range 200-450 mm of Hg. In one version of the embodiment, the prosthesis has a wall thickness in the range of 0.0025-0.0035 inches, or about 64-90 microns, a typical strut width of about 0.002-0.005 inches or about 50-130 microns, typical openings (in the wall of the prosthesis) of around 500 microns, a largest potential opening (for side branch access) of about 2.95 mm and coverage (prosthesis wall member area/total tubular area) of 20% (in its expanded state).

Accordingly, one embodiment of the invention provides a stent or tubular endoluminal prosthesis for the treatment of an atherosclerotic lesion, such as a vulnerable plaque, that includes: a plurality of radial bands comprising a plurality of arch-shaped elements each including a curve portion, two leg portions and two feet (one at the “base” of each leg portion), the arch-shaped elements being arranged in a band and alternating in lateral orientation and being connected to radially neighboring arch-shaped elements by an arch-connecting element that connects to the feet of radially neighboring arch elements; and a plurality of band-connecting elements connecting laterally neighboring radial bands to each other, the band-connecting elements connecting the peak of an arch-element to the trough of a laterally neighboring arch element of a laterally neighboring band. The band connecting element may be disposed in a laterally alternating manner. In another variation, two band-connecting elements are not placed at the same radial position to connect three sequentially positioned radial bands. In still another variation, band-connecting elements may be placed at the same radial positions to continuously connect laterally adjacent radial bands all the way laterally across the stent or prosthesis.

A smooth curve may be formed by the connection of the arch-connecting elements and the feet of neighboring arch elements. The main portion of the arch-connecting elements may, for example, be formed of an at least substantially straight bar element.

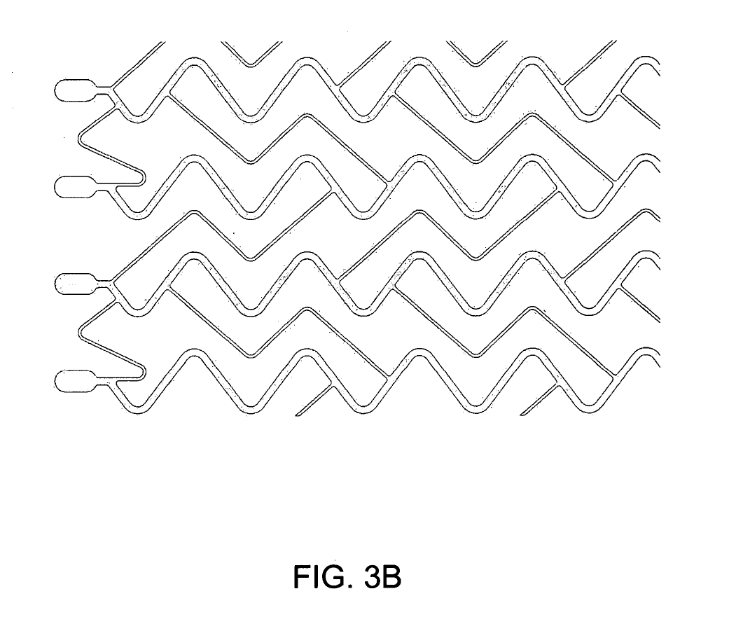

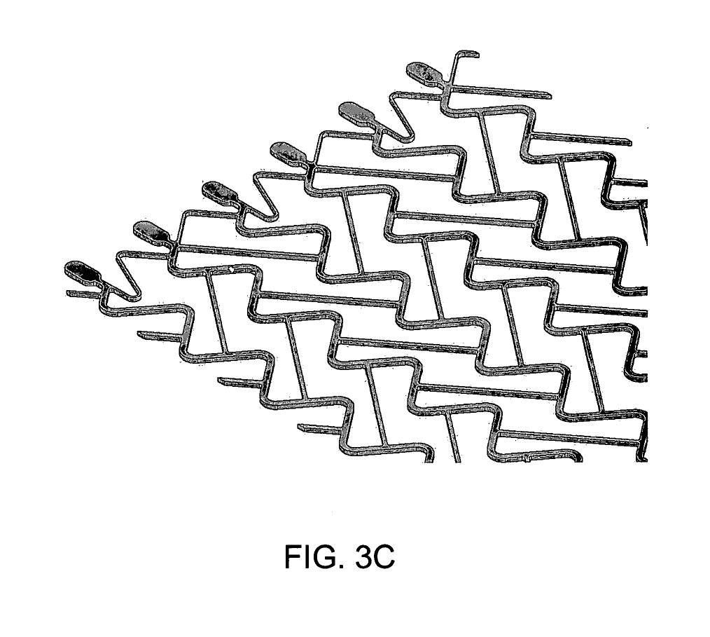

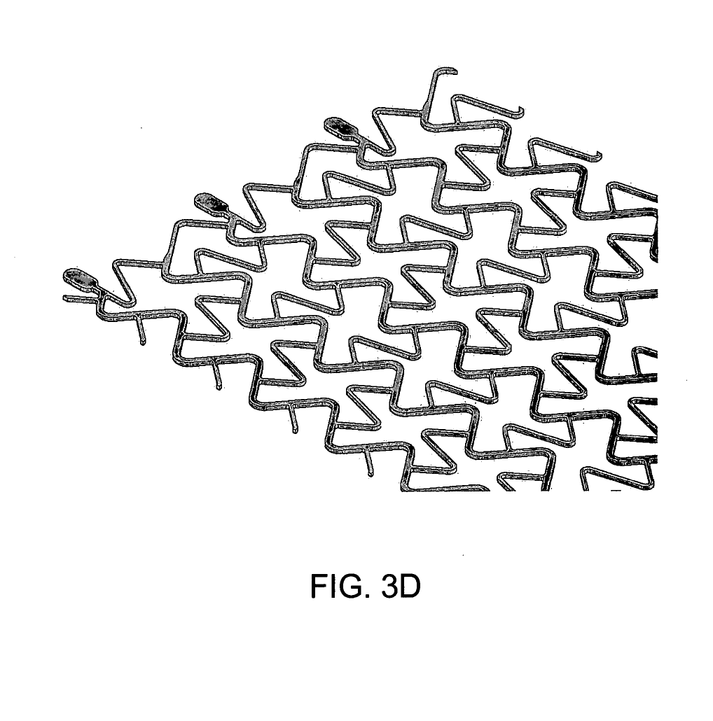

FIG. 3A shows an embodiment of a prosthesis according to the invention that includes longitudinally oriented sinusoidal members (to promote endothelialization) connected by sinusoidal longitudinally-traversing struts members. The view shown in FIG. 3A is a schematic “rolled-out,” flattened view of the tubular configuration. FIG. 3B shows a close-up view of the structure of the embodiment of FIG. 3A. This design exerts very low radial force but did not exhibit optimal conformability for vulnerable plaque use. In one version of the embodiment, the prosthesis has a wall thickness in the range of 0.0025-0.0035 inches, or about 64-90 microns, a typical strut width of about 0.005 inches or about 130 microns, typical openings (in the wall of the prosthesis) of around 500 microns, a largest potential opening (for side branch access) of about 1.44 mm and coverage (prosthesis wall member area/total tubular area) of 18% (in its expanded state).

Accordingly, one embodiment of the invention provides a stent or tubular endoluminal prosthesis for the treatment of an atherosclerotic lesion, such as a vulnerable plaque, having a longitudinal axis and including: a plurality of longitudinally arranged sinusoidal backbone elements that are in-phase; a plurality of backbone-connecting elements that connect radially neighboring backbone elements, wherein the points of connection at the ends of each connecting element to radially neighboring backbone elements are separated by two wavelengths, or approximately so, with respect to the phase of the backbone elements, and wherein the backbone-connecting elements consist of three bar segments oriented to follow the shape of the backbone elements to which connecting element are connected.

The diagonal orientation of the backbone-connecting elements may be uniform laterally but alternate radially with respect to the prosthesis. The points of connection to the backbone elements may occur between a peak and trough of a backbone element to which the connection is made, such as at or about midway between the peak and trough.

At each end of the prosthesis, the backbone elements may each terminate in an atraumatic tab element. The tab element may, for example, have an oval or rounded rectangular configuration having a longitudinal axis that is aligned with the longitudinal axis of the prosthesis.

As shown in FIG. 3B in a radially alternating fashion, some of the connector elements may have a point of contact near the tab elements. As further shown in FIG. 3B special end-connecting elements connect the backbone elements near the tabs for the locations where the backbone-connecting elements are not connected close to the tab (in FIG. 3B, in the cases where the backbone element's point of contact closest to the tab is about 0.5 wavelength from the end of the prosthesis.)

FIG. 3C shows a close-up view of an embodiment of a prosthesis according to the invention that includes longitudinally oriented sinusoidal members (to promote endothelialization) connected by straight longitudinally-traversing struts members. In one version of the embodiment, the prosthesis has a wall thickness in the range of 0.0025-0.0035 inches, or about 64-90 microns, a typical strut width of about 0.002-0.005 inches or about 50-130 microns, typical openings (in the wall of the prosthesis) of around 500 microns, a largest potential opening (for side branch access) of about 1.4 mm and coverage (prosthesis wall member area/total tubular area) of 18-20% (in its expanded state).

Accordingly, one embodiment of the invention provides a stent or tubular endoluminal prosthesis for the treatment of an atherosclerotic lesion, such as a vulnerable plaque, having a longitudinal axis and including: a plurality of longitudinally arranged sinusoidal backbone elements that are in-phase; a plurality of backbone-connecting elements that connect radially neighboring backbone elements, wherein the points of connection at the ends of each connecting element to radially neighboring backbone elements are separated by one wavelength with respect to the phase of the backbone elements, wherein the backbone-connecting elements consist essentially of a single bar segment that may be at least substantially straight.

As shown in the figure, the diagonal orientation of the backbone-connecting elements may be uniform laterally but alternate radially with respect to the prosthesis. The points of connection to the backbone elements may be between, such as about midway between, a peak and trough of a backbone element to which the connection is made. At each end of the prosthesis, the backbone elements may each terminate in an atraumatic tab element. The tab element may for example have an oval or rounded rectangular configuration having a longitudinal axis that is aligned with the longitudinal axis of the prosthesis. The backbone elements are radially interconnected at the ends of the prosthesis by end-connecting elements.

FIG. 3D shows a close-up view an embodiment of a prosthesis according to the invention that includes longitudinally oriented sinusoidal members (to promote endothelialization) connected by sinusoidal radial struts (to enhance radial force and vessel apposition). In one version of the embodiment, the prosthesis has a wall thickness in the range of 0.0025-0.0035 inches, or about 64-90 microns, a typical strut width of about 0.002-0.005 inches or about 50-130 microns, typical openings (in the wall of the prosthesis) of around 500 microns, a largest potential opening (for side branch access) of about 1.4 mm and coverage (prosthesis wall member area/total tubular area) of 18-20% (in its expanded state).

Accordingly, one embodiment of the invention provides a stent or tubular endoluminal prosthesis for the treatment of an atherosclerotic lesion, such as a vulnerable plaque, having a longitudinal axis and including: a plurality of longitudinally arranged sinusoidal backbone elements that are in-phase; a plurality of backbone-connecting elements that connect radially neighboring backbone elements, wherein the backbone-connecting elements consist of a three bar segments (such as a z-shape or mirror image thereof) and wherein the points of connection at the ends of each connecting element to a radially neighboring backbone elements are separated by approximately ¼ wavelength with respect to the phase of the backbone elements.

Again, the orientation of the backbone-connecting elements is uniform laterally but alternates radially with respect to the prosthesis. The points of connection to the backbone elements may occur between, such as approximately midway between, a peak and trough of a backbone element to which the connection is made.

As shown in the figure, at each lateral position at which a radial connecting element is present, a backbone element is only connected to one radially neighboring backbone element, thereby forming a radially alternating pattern of backbone-connecting elements.

As shown, laterally within a row of backbone-connecting elements, said elements are separated by about 1 wavelength from each laterally neighboring backbone-connecting element. The backbone-connecting elements of radially neighboring rows of backbone-connecting elements are laterally offset from one another.

Each of the embodiments shown in FIGS. 3A-3D has atraumatic elliptical structures on the terminal ends of the longitudinal members at each end of the prosthesis. These elliptical “tabs” or “pad shapes” may be folded in half to create “D-shaped” disks that would enhance viewing under fluoroscopic imaging of the shield. Additionally, platinum, iridium and/or tantalum or other radio-dense material may be sandwiched in-between the folded Nitinol disks to further enhance radiopacity. The illustrated end structures are advantageous but are not part of the main-body, structural geometries of the embodiments of FIGS. 3A-3D.

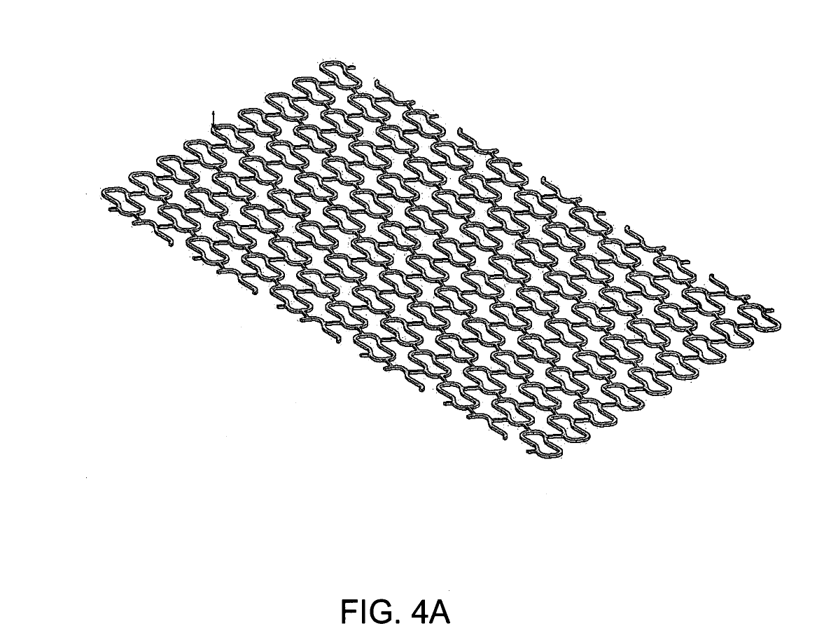

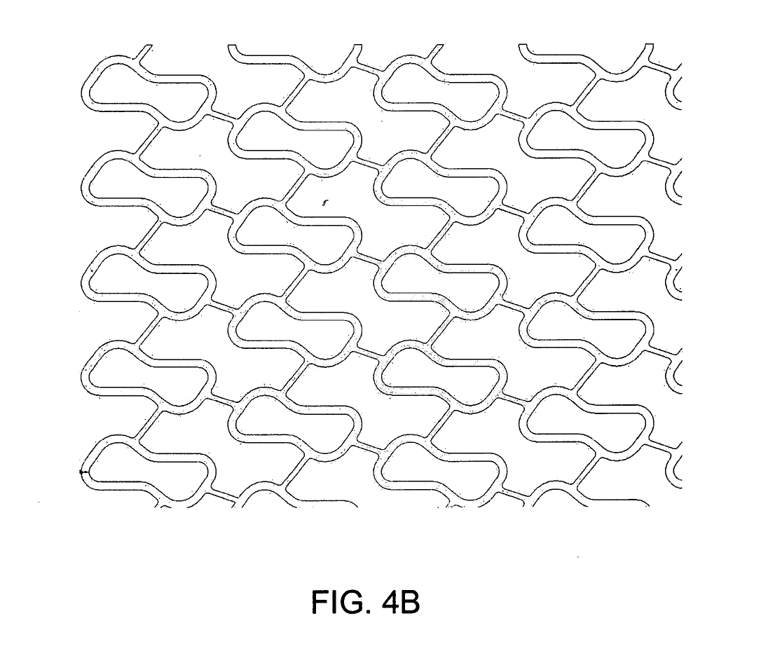

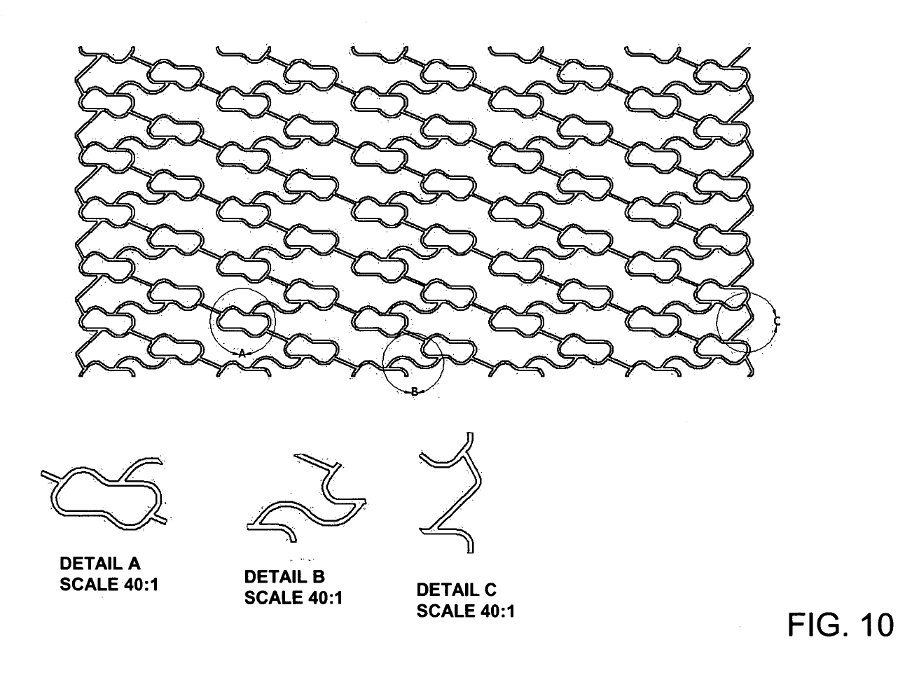

FIG. 4A shows an embodiment of a prosthesis according to the invention that includes compressed elliptical shaped (“hourglass-shaped”) cells disposed at an angle (diagonally) to the longitudinal axis of the prosthesis interconnected by longitudinal and circumferentially oriented struts (to enhance flexibility and radial strength). The view shown in FIG. 4A is a schematic “rolled-out,” flattened view of the tubular configuration. FIG. 4B shows a close-up view of the structure of the embodiment of FIG. 4A. In one version of the embodiment, the prosthesis has a wall thickness in the range of 0.0025-0.0035 inches, or about 64-90 microns, a typical strut width of 0.004 inches or about 100 microns, typical openings (in the wall of the prosthesis) of around 500 microns, a largest potential opening (for side branch access) of about 0.7 mm and coverage (prosthesis wall member area/total tubular area) of about 24% (in its expanded state). This design was found to have a relatively high radial force in tested versions making it less preferred for the treatment of vulnerable plaque lesions. However, the design may nevertheless be used for treating vulnerable plaque lesions by, for example, the selection of metallic or polymeric materials having reduced resilience to decrease hoop strength. A modification of this design is shown in FIG. 10. The design has an increased amount of open area in comparison to the embodiment shown in FIG. 4. Additionally, the compressed hourglass-shaped cells have been thinned out and are connected by “S” shaped struts instead of straight struts. The radial force is thus subsequently reduced, in comparison to the design shown in FIG. 4, to provide further improved treatment of vulnerable plaque. Conformability is also enhanced by these design changes. Coverage has also been reduced, to approximately 15-20%. The use of less material is believed to result in less inflammation and to promote vascular healing.

Accordingly, one embodiment of the invention provides a stent or tubular endoluminal prosthesis for the treatment of an atherosclerotic lesion, such as a vulnerable plaque, having a longitudinal axis and including: a plurality of hourglass-shaped elements (bounded cells) arranged in laterally neighboring radial bands, wherein the longitudinal axes of the hourglass-shaped elements is diagonally oriented with respect to the longitudinal axis of the prosthesis, wherein within a radial band of hourglass-shaped elements each element is connected on it side to a radially neighboring hourglass shaped element by a radial connecting element, and wherein each hourglass-shaped element of a radial band is connected to a hourglass-shaped element of a laterally neighboring radial band that shares the same lateral axis by a lateral connecting element aligned with the lateral (longitudinal) axis of the connected hourglass-shaped elements. The radial connecting elements may be oriented non-perpendicularly with respect to the longitudinal axis of the prosthesis. The radial connecting elements may, for example, include or consist of at least substantially straight bar elements.

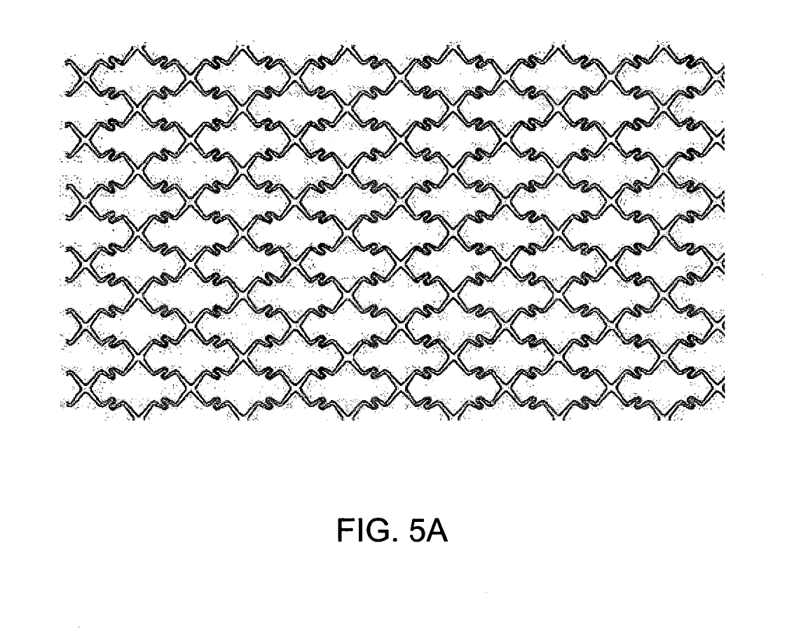

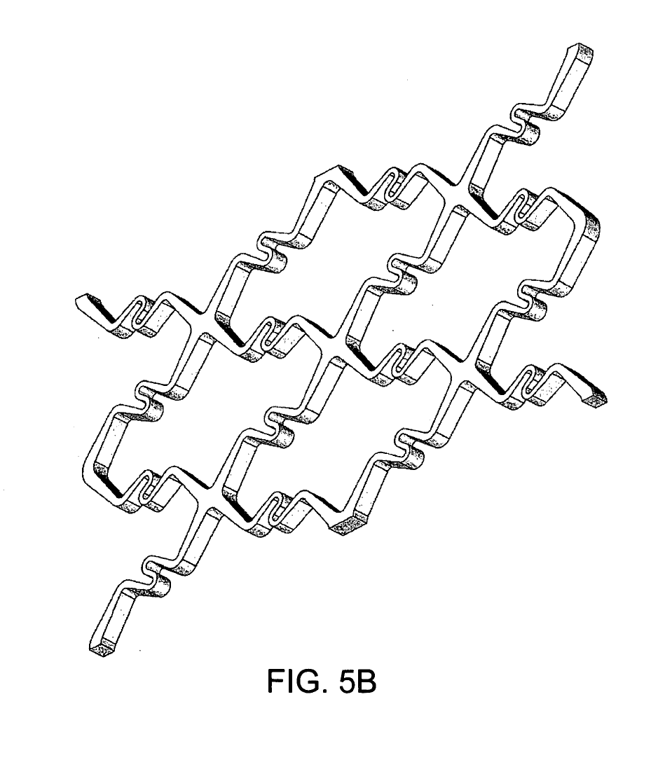

FIG. 5A shows an embodiment of a prosthesis according to the invention that includes X-shaped elements interconnected by smaller sinusoidal connecting elements. The cross-bars of the X-shaped elements are disposed diagonally with respect to the longitudinal axis of the prosthesis. This design is unique in that the overall pattern and behavior of the design resembles that of a braided stent yet it does not exhibit the shortcomings of braided stents such as foreshortening and non-conformability. Additionally, it can be fabricated by laser cutting from a tube or sheet and welded together, or manufactured by other methods. The ends of the design are also terminated by semicircular curves that again give it significant advantages over thin braided designs which typically have individual wires that can become unbraided and potentially move into the vessel lumen. Lastly, the sinusoidal elements minimize foreshortening. The view shown in FIG. 5A is a schematic “rolled-out,” flattened view of the tubular configuration. FIG. 5B shows a close up view of a section of the prosthesis structure of the embodiment of FIG. 5A.

Accordingly, one embodiment of the invention provides a stent or tubular endoluminal prosthesis for the treatment of an atherosclerotic lesion, such as a vulnerable plaque, that includes: a main body portion between the ends of the stent or prosthesis including or consisting essentially of x-shaped structural elements having four corners (the ends of each “cross-bar” that forms the x-shape element) and small undulating connecting elements, wherein each x-shaped element is connected at each of its corners to the corner of one other x-shaped element by a small undulating connecting element. In one variation, at least some of the small undulating connecting elements may be sinuate. In a related variation, at least some of the small undulating connecting elements may be s-shaped. In another variation, at least some of the small undulating connecting elements may be z-shaped.

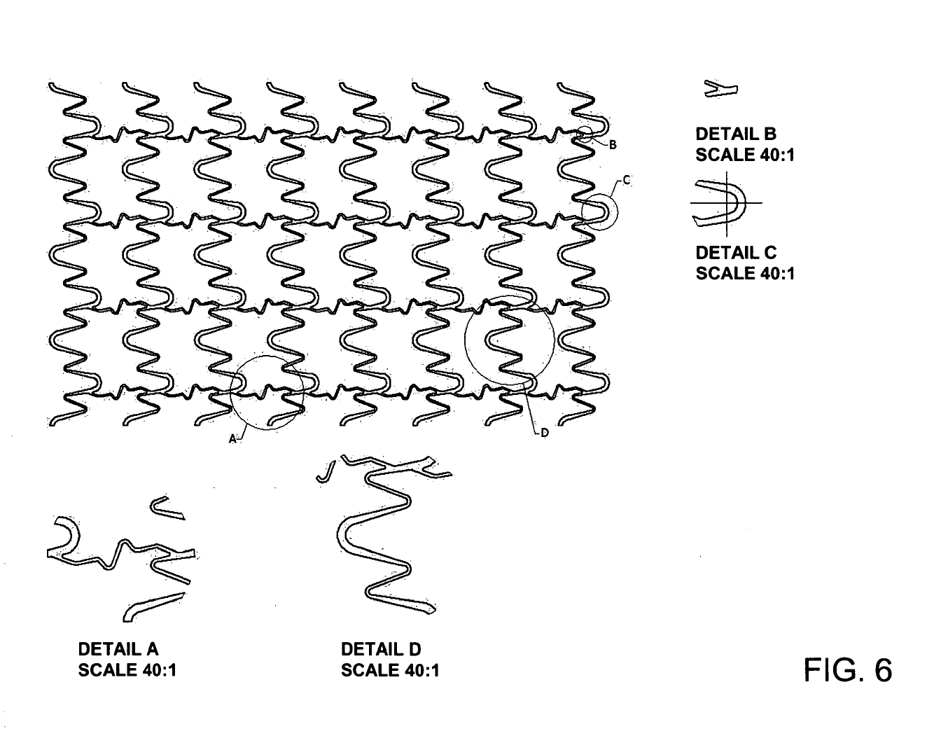

FIG. 6 shows a section of an embodiment of a prosthesis according to the invention that includes sinusoidal ring sections for radial support (see Detail D) interconnected by lateral sinusoidal struts having six bends (inflection points; see Detail A) positioned on an angle from the longitudinal axis. The lateral sinusoidal struts provide column strength for loading into the delivery system and to minimize foreshortening during deployment. The lateral struts also add torsional rigidity that is not provided by linear struts.

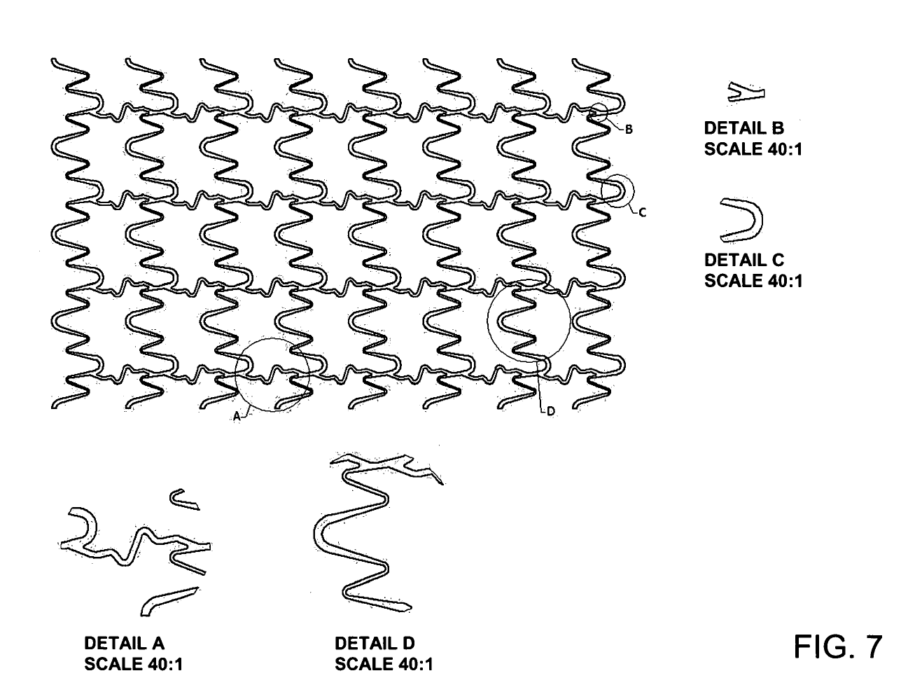

FIG. 7 shows a portion of an embodiment of a prosthesis that is similar to the embodiment shown in FIG. 6. This structure has a more open design, providing a coverage (prosthesis wall member area/total tubular area) of 15% (in its expanded state).

Accordingly, one embodiment of the invention provides a stent or tubular endoluminal prosthesis for the treatment of an atherosclerotic lesion, such as a vulnerable plaque, that includes: a plurality of radial sinusoidal bands each sinusoidal band including peaks and troughs, wherein the peaks and troughs of laterally neighboring bands are in-phase thereby forming rows of arch-elements; a plurality of lateral connector elements connecting neighboring radial bands to each other, wherein the lateral connector elements connect laterally neighboring arch-elements to each other and wherein the lateral connector elements are present in alternating rows of the arch-elements. In rows of arch elements in which the lateral connector elements are present, the lateral connector elements may connect all neighboring radial band elements. The lateral connector elements may connect the arch elements at their peaks to corresponding troughs in laterally neighboring radial bands or the lateral connector elements may connect to arch-elements at points within the leg section (between the peak and foot of one side of an arch element) of the arch elements, for example, as shown in FIGS. 6 and 7. As shown in the figures, radially neighboring arch elements alternate in their lateral orientation and the feet of radially neighboring arch elements are connected to each other by a bar element. There may be a curved, turn-portion (as shown) where a bar element connects to the foot of an arch element, or there may be no curved portion. The legs of neighboring arch elements laterally overlap one another so that the lateral connector elements are diagonally oriented with respect to the longitudinal axis of the prosthesis. The lateral connector elements shown have six inflection points.

The lateral connector elements may be straight or be of a sinuate form, for example, as shown in FIGS. 6 and 7. FIGS. 6, 7, 11 and 13 show embodiments with lateral connector elements of varying shapes and having varying numbers of inflection points. The lateral connector elements may, for example, include one or more segments that are, at least substantially sinusoidal, sinuate, s-shaped, and/or z-shaped.

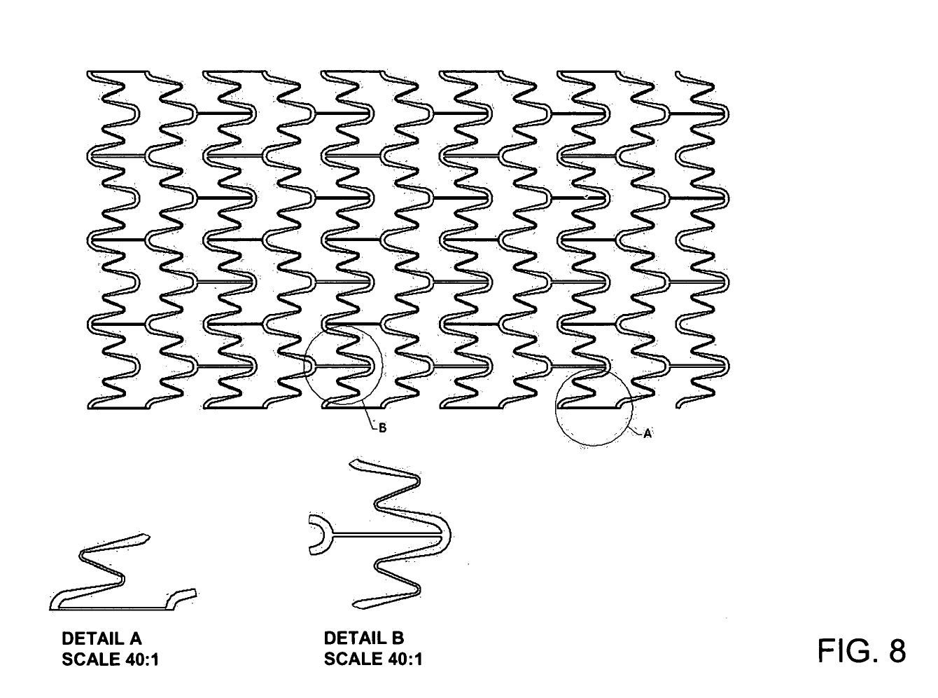

FIG. 8 shows a section of an embodiment of a prosthesis according to the invention that includes circumferential undulating sinusoidal members connected by linear longitudinal struts. The embodiment of FIG. 8 is related to the embodiments of FIGS. 2A and 2B.

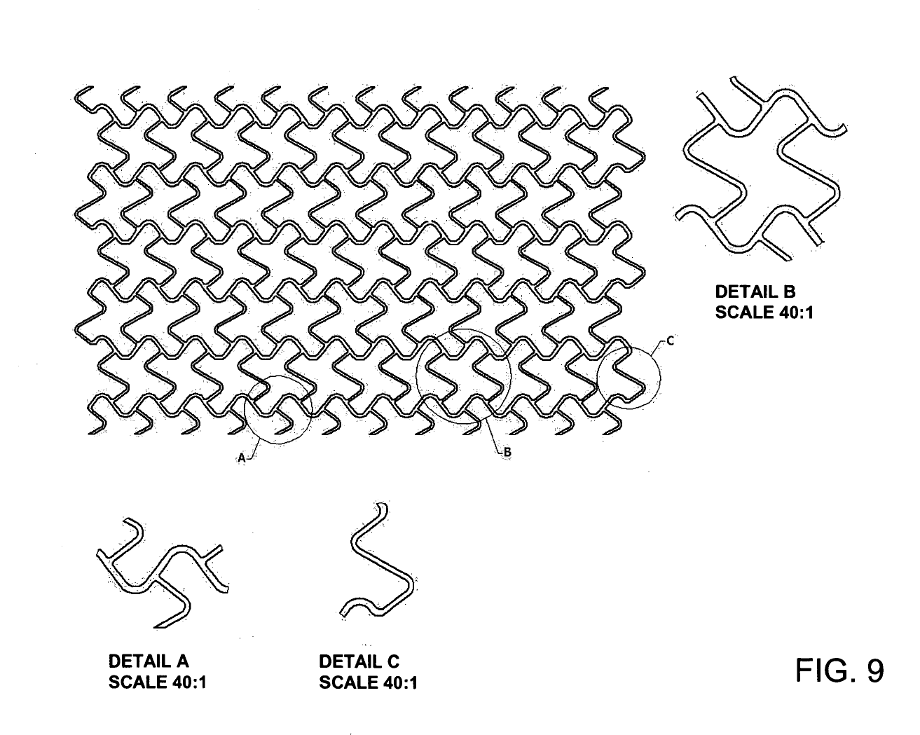

FIG. 9 shows a section of an embodiment of a prosthesis according to the invention that has nested cruciform shaped cells that are formed from lateral (along the longitudinal axis of the prosthesis) sinusoidal elements interconnected by staggered transverse sinusoidal connecting struts which have two bends. A special end structure is also shown at each end of the prosthesis.

Accordingly, one embodiment of the invention provides a stent or tubular endoluminal prosthesis for the treatment of an atherosclerotic lesion, such as a vulnerable plaque, having a longitudinal axis and including: a plurality of longitudinally arranged sinusoidal backbone elements that are in-phase; a plurality of backbone-connecting elements that connect radially neighboring backbone elements, wherein the backbone-connecting elements consist of a three bar segments, for example, in a z-configuration or mirror-image thereof, and wherein the points of connection at the ends of each connecting element to a radially neighboring backbone elements are at least approximately in phase with respect to the phase of the backbone elements.

As shown in the figure, the orientation of the backbone-connecting elements may be uniform laterally but alternate radially with respect to the prosthesis. The points of connection to the backbone elements may occur between, such as about midway between, a peak and trough of a backbone element to which the connection is made.

At each lateral position at which a radial connecting element is present, a backbone element is only connected to one radially neighboring backbone element, thereby forming a radially alternating pattern of backbone-connecting elements. The backbone-connecting elements of radially neighboring rows of backbone-connecting elements may be laterally offset from one another.

FIG. 10 shows an embodiment of a prosthesis according to the invention that includes compressed elliptical shaped (“hourglass-shaped”) cells disposed at an angle (diagonally) to the longitudinal axis of the prosthesis in which the ends of adjacent hour-glass shaped elements are connected by straight struts (aligned with the lateral axes of the hourglass-shaped cells) and each hourglass-shaped element is connected at its side to one transversely adjacent hourglass-shaped element by a sinusoidal connecting element. A special end structure is also shown at each end of the prosthesis in which the end-face of each hourglass-shaped element is connected to the side of the transversely adjacent hourglass-shaped element by a connecting element having a single bend. This embodiment has a radial force that is sufficiently low to treat vulnerable plaque. Additionally, the interconnecting sinusoids and reduced strut thickness of the compressed hourglass-shaped cells enhance conformability.

Accordingly, one embodiment of the invention provides a stent or tubular endoluminal prosthesis for the treatment of an atherosclerotic lesion, such as a vulnerable plaque, that includes: a plurality of hourglass-shaped elements (bounded cells) arranged in laterally neighboring radial bands, wherein the longitudinal axes of the hourglass-shaped elements are diagonally oriented with respect to the longitudinal axis of the prosthesis, wherein each hourglass-shaped element of a radial band is connected to an hourglass-shaped element of a laterally neighboring radial band that shares the same lateral axis by a lateral connecting element aligned with the lateral axis of the connected hourglass-shaped elements, and wherein each hourglass-shaped element is connected to a different, non-axially-coaligned, hourglass-shaped element of a laterally neighboring radial band by a sinuate connecting element attached to the side of each of the hourglass-shaped elements so connected. The sinuate connecting element may, for example be s-shaped or sinusoidal.

The side-to-side connection of the hourglass shaped elements by a sinuate connecting element may, for example, occur at points of connection at or near the waists (point of narrowing) of the sinuate element connected hourglass-shaped elements.

It can be seen that in this embodiment, except optionally at the ends of the prosthesis, the hourglass-shaped elements in a radial band of hourglass shaped elements are not directly radially connected to radially neighboring hourglass-shaped elements.

The hourglass-shaped elements present at the ends of the prosthesis may, for example be connected to radially neighboring hourglass-shaped elements by radial end-connecting elements that connect the outward facing ends of the hourglass shaped element to the a point on the side, such as at or near the waist of radially neighboring hourglass-shaped elements, for example, as shown in FIG. 10.

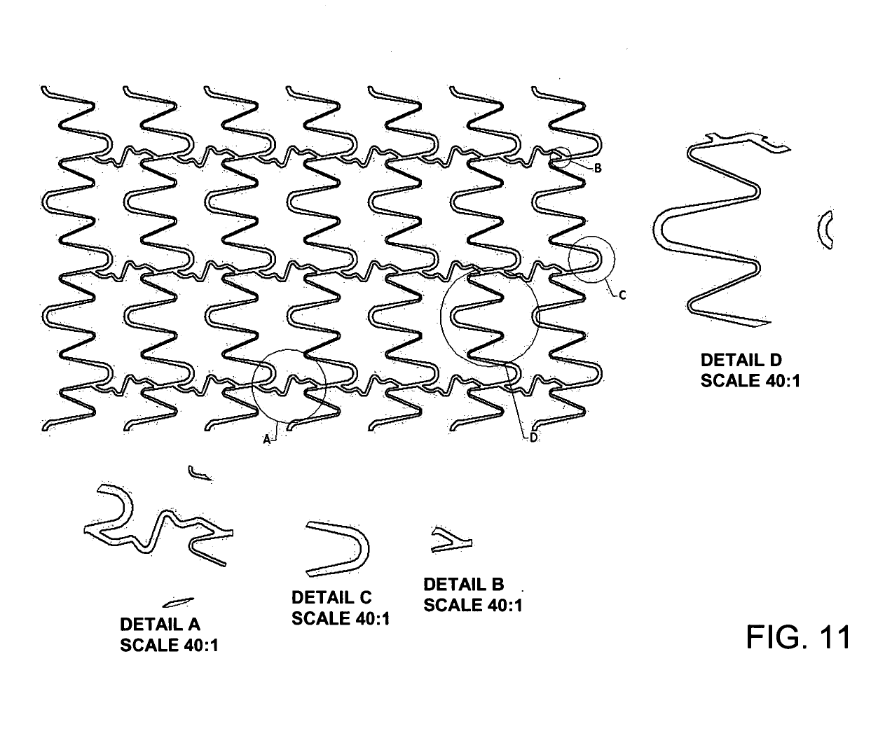

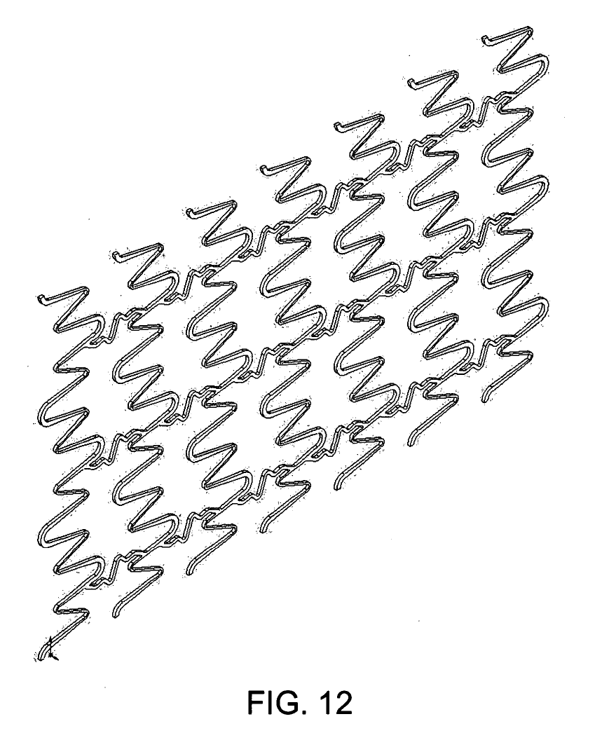

FIGS. 11 (flat pattern) and 12 (isometric view) show a portion of an embodiment of a prosthesis that is similar to the embodiment shown in FIG. 6. This structure has an even more open design, providing a coverage (prosthesis wall member area/total tubular area) of 11% (in its expanded state). Additionally, this design will collapse to an even smaller diameter for delivery.

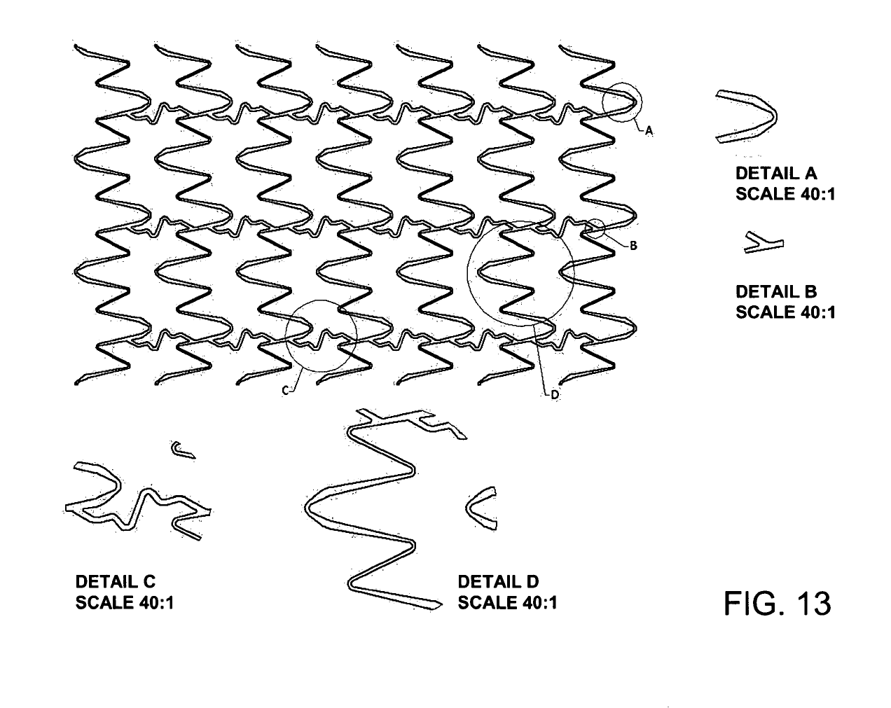

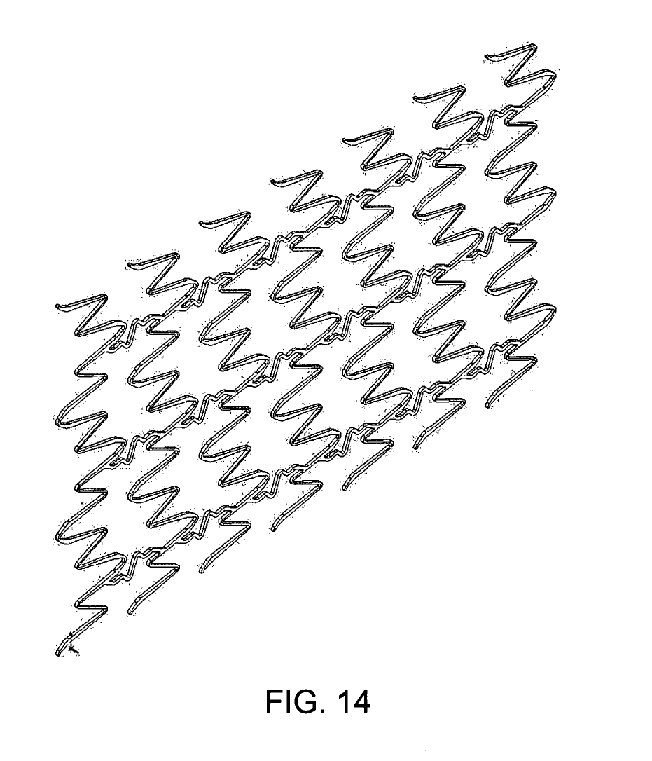

FIGS. 13 (flat pattern) and 14 (isometric view) shows an embodiment that is similar to that shown in FIGS. 6 and 11, but including arch-elements having a different contour shape. Specifically, the part of the arch-elements at and immediately surrounding the peaks of the arch elements in FIG. 13 is narrowed versus the embodiment shown in FIG. 11. Thus, a hinge feature, in the form of a narrowing of width, has been provided in this embodiment (see Detail A). The hinge feature allows the structure to collapse to an even as smaller diameter for loading into the delivery system. The structure of FIG. 13 also has an open design, providing a coverage (prosthesis wall member area/total tubular area) of approximately 11% (in its expanded state). Hence, the profile of the delivery system can be even smaller to facilitate access to the coronary arteries. The hinge feature also adds more flexibility to the overall structure to enhance vessel wall conformability in its expanded state and to enhance delivery system flexibility in its compressed state.

A further embodiment of the invention provides a method for treating an atherosclerotic lesion, such as a vulnerable plaque, in a patient in need thereof that includes the step of deploying any of the prostheses described herein at the site of the lesion in the patient. Preferably, the device is positioned so that it at least partially traverses a section of blood vessel that has the atherosclerotic lesion. The deployment involves an expansion of the radius of the device to that the end sections and the strut sections come into contact with the vessel wall. For treatment of vulnerable plaques, at least one of the strut sections may contact the fibrous cap of the vulnerable plaque and/or at least one strut section may contact the vessel wall in the vicinity of the vulnerable plaque lesion. In either case, contact with the vessel wall promotes endothelialization and remodeling of at least the luminal face of the vulnerable plaque lesion. The prostheses of the invention may be delivered in a decreased radius configuration on a delivery catheter. The prostheses may be crimped on or otherwise positioned around an inflatable deployment balloon, so that expansion of the balloon at least partially expands the prosthesis to its final working radius. For self-expanding versions of a prosthesis according to the invention, use of a delivery balloon is optional. A self-expanding prosthesis may, for example, be restrained in a cylindrical cavity covered by a restraining sheath and deployed by retracting the sheath, as known in the art.

The prostheses of the invention may, for example, be sized for catheter delivery into, and deployment in (expansion to contact vessel wall/lesion), human coronary arteries, thus, sized for the treatment of human coronary arteries.

Any of the treatment methods of the invention may include a step of locating an atherosclerotic lesion, such as a vulnerable plaque lesion, to be treated by the prosthesis in a patient.

According to the invention, determining the location of a vulnerable plaque in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differences characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated herein in its entirety. A method and system for detecting and localizing vulnerable plaques based on the detection of biomarkers is disclosed in U.S. Pat. No. 6,860,851, which is hereby incorporated by reference herein in its entirety. Time-resolved laser-induced fluorescence spectroscopy (TR-LIFS) may also be used to detect and locate vulnerable plaques. U.S. Pat. No. 6,272,376 teaches TR-LIFS methods for detecting lipid-rich vascular lesions and is hereby incorporated by reference herein in its entirety.

Angiography using a radiopaque and/or fluorescent dye, for example, as known in the art, may be performed before, during and/or after the step of determining the location of the vulnerable plaque, for example, to assist in positioning the prosthesis in a subject artery or other blood vessel.

The prostheses of the invention may be metallic and/or polymeric in composition.

Metals used to manufacture a prosthesis according to the invention include, but are not limited to stainless steel, titanium, titanium alloys, platinum and gold. Shape-memory metal alloys may be used to produce self-expanding versions of prostheses according to the invention. For example, suitable shape-memory alloys include, but are not limited, to Nitinol and Elgiloy.

Polymers used for the manufacture of prostheses according to the invention may be biodegradable or non-biodegradable. Any suitable sorts of biodegradable polymers and/or biodegradable polymer blends may be used according to the invention. As used herein, the term “biodegradable” should be construed broadly as meaning that the polymer(s) will degrade once placed within a patient's body. Accordingly, biodegradable polymers as referred also include bioerodable and bioresorbable polymers. Suitable types of polymer material include, but are not limited to, polyester, polyanhydride, polyamide, polyurethane, polyurea, polyether, polysaccharide, polyamine, polyphosphate, polyphosphonate, polysulfonate, polysulfonamide, polyphosphazene, hydrogel, polylactide, polyglycolide, protein cell matrix, or copolymer or polymer blend thereof.

Homopolymers of polylactic acid (PLA), for example PLLA, PDLA and poly(D,L,)lactic acid, stereopolymers thereof, and copolymer of PLA with other polymeric units such as glycolide provide a number of characteristics that are useful in a polymeric prosthesis for treating a lesion of a blood vessel such as a high risk atherosclerotic plaque (vulnerable plaque). First, polymers made of these components biodegrade in vivo into harmless compounds. PLA is hydrolyzed into lactic acid in vivo. Second, these polymers are well-suited to balloon-mediated expansion using a delivery catheter. Third, polymers made of these materials can be imparted with a shape-memory so that polymeric, at least partially self-expanding, tubular prostheses can be provided. Self-expanding polymeric prostheses according to the invention may also, for example, be at least partially balloon-expanded. Methods for producing biodegradable, polymeric shape-memory prostheses are described, for example, in U.S. Pat. Nos. 4,950,258, 5,163,952, and 6,281,262 each of which is incorporated by reference herein in its entirety.

Prostheses according to the invention may be manufactured by any suitable method. For example, a metallic prosthesis can be produced by laser cutting the device from a tubular blank. Methods for forming metallic tubular blanks are well known. For example, sputtering metallic material onto a mandrel may be used. In another example, the shape of the prosthesis can be laser cut or stamped out of a flat sheet of metallic material and then formed and welded into a tubular configuration. Once formed into shape, metallic prostheses according to the invention may optionally be electrochemically polished and/or etched.

The wall thickness of an prosthesis according to the invention may, for example, be in the range of about 20 microns to about 200 microns. In one embodiment, the wall thickness is equal to or less than 200 microns, for example, equal to or less than 125 microns. In one embodiment, the wall thickness is in the range of 20 microns to 125 microns. In another embodiment of the invention, the wall thickness is in the range of 20 to 60 microns. In still another embodiment, the wall thickness is in the range of 50 to 100 microns.

A polymeric prosthesis according to the invention, such as one composed of polylactide, may also be laser cut from a tubular blank, such as one formed by extrusion molding.

Prostheses according to the invention may optionally be provided with a polymeric, metallic or composite cover that surrounds at least part of the strut sections of the prosthesis. In one embodiment, irrespective of the composition of the body of the prosthesis, the cover may be polymeric and may, for example, be biodegradable in vivo. The polymer cover may be self-expanding, for example as the result of a shape-memory characteristic. The cover may, for example, be thermoplastically expandable but not be self-expanding. The cover may be porous or non-porous. The cover may, for example, be a continuous porous or non-porous polymeric structure or it may be a braid, woven, or knit polymeric structure. In embodiment in which at least a portion of the strut section is covered, the cover rather than the underlying struts contact the vessel wall upon deployment of the device.

For polymeric prostheses, it may also be possible to blend one or more beneficial agents such as drugs with the polymer melt during the formation of an article. Metallic or non-metallic prostheses according to the invention may be coated with one or more polymer coatings. The coating(s) may optionally include or be loaded with beneficial agents such as drugs or other compounds useful for treating vulnerable and/or for facilitating the desired functioning of the implanted prosthesis, for example, anti-thrombotic agents such as heparin to inhibit prosthesis-induced thrombosis at the treatment site. U.S. Pat. No. 5,624,411 teaches methods of coating intravascular stents with drugs, and is hereby incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

1-13. (canceled)

14. A tubular endoluminal prosthesis for the treatment of an atherosclerotic lesion having a longitudinal axis and comprising:
a plurality of longitudinally arranged sinusoidal backbone elements that are in-phase;
a plurality of backbone-connecting elements that connect radially neighboring backbone elements, wherein the points of connection at the ends of each connecting element to radially neighboring backbone elements are separated by two wavelengths with respect to the phase of the backbone elements, and
wherein each backbone-connecting elements consists of three bar segments oriented to follow the shape of the backbone elements to which connecting element are connected.

15. The prosthesis of claim 14, wherein the backbone-connecting elements are diagonally oriented with respect to the longitudinal axis and the diagonal orientation of the backbone-connecting elements is uniform laterally and alternates radially with respect to the prosthesis.

16. The prosthesis of claim 14, wherein the points of connection to the backbone elements is between a peak and trough of a backbone element to which the connection is made.

17. The prosthesis of claim 14, wherein at each end of the prosthesis, the backbone elements each terminate in an atraumatic tab element.

18-21. (canceled)

22. A tubular endoluminal prosthesis for the treatment of an atherosclerotic lesion having a longitudinal axis, comprising:
a plurality of longitudinally arranged sinusoidal backbone elements that are in-phase:
a plurality of backbone-connecting elements that connect radially neighboring backbone elements,
wherein the backbone-connecting elements consist of three bar segments and
wherein the points of connection at the ends of each connecting element to a radially neighboring backbone elements are separated by approximately ¼ wavelength with respect to the phase of the backbone elements.

23. The prosthesis of claim 22, wherein the orientation of backbone-connecting elements is uniform laterally and alternates radially with respect to the prosthesis.

24. The prosthesis of claim 22, wherein the points of connection to the backbone elements is between a peak and trough of a backbone element to which the connection is made.

25. The prosthesis of claim 22, wherein at each end of the prosthesis, alternating backbone elements terminate in an atraumatic tab element.

26. The prosthesis of claim 22, wherein, except optionally at the ends of the prosthesis, at each lateral position at which a radial connecting element is present, a backbone element is only connected to one radially neighboring backbone element, thereby forming a radially alternating pattern of backbone-connecting elements.

27. The prosthesis of claim 22, wherein laterally within a row of backbone-connecting elements, said elements are separated by about one wavelength from each laterally neighboring backbone-connecting element.

28. The prosthesis of claim 22, wherein the backbone-connecting elements of radially neighboring rows of backbone-connecting elements are laterally offset from one another.

29. A tubular endoluminal prosthesis for the treatment of an atherosclerotic lesion having a longitudinal axis and comprising:
a plurality of longitudinally arranged sinusoidal backbone elements that are in-phase;
a plurality of backbone-connecting elements that connect radially neighboring backbone elements,
wherein the backbone-connecting elements consist of a three bar segments having a z-configuration or mirror-z-configuration, and
wherein the points of connection at the ends of each backbone-connecting element to a radially neighboring backbone element are at least approximately in phase with respect to the phase of the backbone elements.

30. The prosthesis of claim 29, wherein the orientation of backbone-connecting elements is uniform laterally and alternates radially with respect to the prosthesis.

31. The prosthesis of claim 29, wherein the points of connection to the backbone elements is between a peak and trough of a backbone element to which the connection is made.

32. The prosthesis of claim 29, wherein at each lateral position at which a backbone-connecting element is present, a backbone element is only connected to one radially neighboring backbone element, thereby forming a radially alternating pattern of backbone-connecting elements.

33. The prosthesis of claim 29, wherein laterally within a row of backbone-connecting elements, said elements are separated by about 0.5 wavelength from each laterally neighboring backbone-connecting element.

34. The prosthesis of claim 29, wherein the backbone-connecting elements of radially neighboring rows of backbone-connecting elements are laterally offset from one another.

35-41. (canceled)

42. A tubular endoluminal prosthesis for the treatment of an atherosclerotic lesion having two ends and comprising:
a main body portion disposed between the ends of the prosthesis that consists essentially of x-shaped structural elements having four corners and small undulating connector elements,
wherein each x-shaped element is connected at each of its corners to the corner of one other x-shaped element by a small undulating connector element.

43. The prosthesis of claim 42, wherein, the small undulating connecting elements comprise connecting elements that arc sinuate in form.

44. The prosthesis of claim 42, wherein, the small undulating connecting elements comprise connecting elements that are s-shaped.

45. The prosthesis of claim 42, wherein, the small undulating connecting elements comprise connecting elements that are z-shaped.

46-50. (canceled)

51. A method for treating vulnerable plaque in a patient in need thereof, comprising the steps of:
deploying a prosthesis according to claim 14 at a site of a vulnerable plaque in a blood vessel of a patient.

52-53. (canceled)

54. A method for treating vulnerable plaque in a patient in need thereof, comprising the steps of:
deploying a prosthesis according to claim 22 at a site of a vulnerable plaque in a blood vessel of a patient.

55. A method for treating vulnerable plaque in a patient in need thereof, comprising the steps of:
deploying a prosthesis according to claim 29 at a site of a vulnerable plaque in a blood vessel of a patient.

56. A method for treating vulnerable plaque in a patient in need thereof, comprising the steps of:
deploying a prosthesis according to claim 42 at a site of a vulnerable plaque in a blood vessel of a patient.
